(19) United States
(12) Patent Application Publication
(10) Pub. No.: US 2009/0180978 A1
(43) Pub. Date: Jul. 16, 2009

(54) Cosmetic composition

(76) Inventors: Nathalie Schlemer, Culver City, CA (US); Nick Morante, Selden, NY (US)

Correspondence Address: Cislo & Thomas LLP, 1333 2nd Street, Suite #500, Santa Monica, CA, 90401-4110, US

(21) Appl. No.: 12/317,439

(22) Filed: Dec. 22, 2008

Related U.S. Application Data
(60) Provisional application No. 61/009,543, filed on Dec. 27, 2007.

(51) Int. Cl. A61K 8/18; A61K 8/97; A61Q 1/02

(52) U.S. Cl. 424/63; 424/725

(57) ABSTRACT

The present invention provides a cosmetic composition ideally formulated as a make-up concealer including the following ingredients: caprylic/capric triglyceride, octyldodecanol, euphorbia cerifera (candelilla) wax, copernicia cerifera (camauba) wax, jojoba esters, polyethylene, stearalkonium bentonite, dimethicone, allantoin, tocopheryl acetate, ascorbyl palmitate, titanium dioxide, and iron oxides.

### CROSS REFERENCE TO RELATED APPLICATIONS

This document is a U.S. Utility Patent Application which is related to, and claims the priority through earlier filed U.S. Provisional Application Ser. No. 61/009,543, filed on Dec. 27, 2007 all the subject matter of which is herein incorporated by this reference thereto in its entirety for all purposes.

### BACKGROUND

1. Field of the Invention

The present invention relates to cosmetic compositions, and particularly cosmetic concealers.

2. Description of the Related Art

Various cosmetic products are used to improve the appearance of the skin. Such products include concealers which match the skin shade, and provide coverage for skin blemishes and other imperfections. As such, there is always a need for improved products, such as concealers, and for products which are beneficial and non-irritating to the skin, and which provide improved aesthetic results.

### SUMMARY

The present invention provides a cosmetic composition ideally formulated as a make-up concealer including the following ingredients: caprylic/capric triglyceride, octyldodecanol, euphorbia cerifera (candelilla) wax, copernicia cerifera (camauba) wax, jojoba esters, polyethylene, stearalkonium bentonite, dimethicone, allantoin, tocopheryl acetate, ascorbyl palmitate, titanium dioxide, and iron oxides.

### DETAILED DESCRIPTION

The detailed description set forth below is intended as a description of an exemplary embodiment and is not intended to represent the only forms in which the exemplary embodiment may be constructed and/or utilized. The description sets forth the functions and the sequence of steps for constructing and/or operating the exemplary embodiment. However, it is to be understood that the same or equivalent functions and sequences may be accomplished by different exemplary methods are also intended to be encompassed within the spirit and scope of the invention.

As used herein, “safe and effective amount” means a sufficient amount of a compound, composition or other material described by this phrase to significantly induce a positive modification in the condition being treated, but low enough to avoid undue side effects (e.g., significant skin irritation or sensitization) within the scope of sound judgment of the skilled person. The safe and effective amount of the compound, composition or other material may vary with the particular skin being treated, the age and physical condition of the biological subject being treated, the severity of the condition, the duration of treatment, the nature of concurrent therapy, the specific compound, composition, or other material employed, the particular cosmetically acceptable topical carrier utilized, and the factors within the knowledge and expertise of the skilled person.

The formulation of the present invention is a concealer, comprising emulsifying agents including octylodecanol and caprylic/capric triglyceride, waxes including candelilla and carnauba wax, moisturizers and anti-irritants including jojoba esters and allantoin, skin protectants including dimethicone, tocopheryl acetate, ascorbyl palmitate, and visual effect agents comprising mica and iron oxide. Other essential ingredients are listed in the tables for examples provided below.

The following are examples for the preparation of a make-up concealer, in accordance with a preferred embodiment of the present invention.

The table below provides an example of a concealer having a “light” shade.

| Trade Name | INCI Name | % Wt. | Supplier |
|---|---|---|---|
| (A) Jarcol I20 | Octylodecanol | 3.600 | Jarchem |
| (A) Candelilla Wax Regular | Euphorbia Cerifera (Candelilla) | 3.140 | Koster- |
| | Wax | | Keunen |
| (A) Carnauba Wax #1 | Copernica Cerifera (Carnauba) | 3.140 | Koster- |
| | Wax | | Keunen |
| (A) Floraesters 30 | Jojoba Esters | 2.510 | Floratech |
| | | | America |
| (A) DC 200/100 Fluid | Dimethicone | 0.090 | Dow Corning |
| (A) Vitamin E Acetate | Tocopheryl Acetate | 0.090 | BASF |
| (A) Allantoin | Allantoin | 0.050 | ISP |
| | | | Corporation |
| (A) Ascorbyl Palmitate | Ascorbyl Palmitate | 0.050 | DSM |
| NF-FCC | | | Nutritionals |
| (A) Polyethylene 6A | Polyethylene | 1.210 | Honeywell |
| (A) Tixogel CCT6030 | Caprylic/Capric Triglyceride and | 5.140 | Sod-Chemie |
| | Stearalkonium-90 | | Rheologicals |
| | Bentonite and Propylene | | |
| | Carbonate | | |
| (B) Sericite PHN | Mica | 7.300 | Presperse |
| (B) Jarcol 120 | Octyldodecanol | 2.420 | Jarchem |
| (B) Emeressence 1160 | Phenoxyethanol | 1.000 | Cognis |
| (B) SensivaSC50 | Ethylhexylglycerin | 1.000 | Schulke & |
| | | | Mayer |
| (C) Liponate GC | Caprylic/Capric Triglyceride | 42.296 | Lipo |
| | | | Chemicals |
| (C) Atlas White 28190 Oil | Titanium Dioxide (77491) | 24.310 | LCW |
| Dispersible Lot #W05280 | | | |
| (C) Unipure Red LC381 | Iron Oxide (CI77491) | 0.227 | LCW |
| Lot #B7259 | | | |
| (C) Unipure Yellow LC181 | Iron Oxide (CI77492) | 2.400 | LCW |
| Lot #B1946 | | | |
| (C) Unipure Black LC989 | Iron Oxide (CI77499) | 0.027 | LCW |
| Lot #B1273 | | | |

The table below provides an example of a concealer having a “medium” shade.

| Trade Name | INCI Name | % Wt. | Supplier |
|---|---|---|---|
| (A) Jarcol I20 | Octylodecanol | 3.600 | Jarchem |
| (A) Candelilla Wax Regular | Euphorbia Cerifera (Candelilla) | 3.140 | Koster- |
| | Wax | | Keunen |
| (A) Carnauba Wax #1 | Copernica Cerifera (Carnauba) | 3.140 | Koster- |
| | Wax | | Keunen |
| (A) Floraesters 30 | Jojoba Esters | 2.510 | Floratech |
| | | | America |
| (A) DC 200/100 Fluid | Dimethicone | 0.090 | Dow Corning |
| (A) Vitamin B Acetate | Tocopheryl Acetate | 0.090 | BASF |
| (A) Allantoin | Allantoin | 0.050 | ISP |
| | | | Corporation |
| (A) Ascorbyl Palmitate | Ascorbyl Palmitate | 0.050 | DSM |
| NF-FCC | | | Nutritionals |
| (A) Polyethylene 6A | Polyethylene | 1.210 | Honeywell |
| (A) Tixogel CCT6030 | Caprylic/Capric Triglyceride and | 5.140 | Sod-Chemie |
| | Stearalkonium-90 | | Rheologicals |
| | Bentonite and Propylene | | |
| | Carbonate | | |
| (B) Sericite PHN | Mica | 7.300 | Presperse |
| (B) Jarcol 120 | Octyldodecanol | 2.420 | Jarchem |
| (B) Emeressence 1160 | Phenoxyethanol | 1.000 | Cognis |
| (B) SensivaSC50 | Ethylhexylglycerin | 1.000 | Schulke & |
| | | | Mayer |
| (C) Liponate GC | Caprylic/Capric Triglyceride | 41.695 | Lipo |
| | | | Chemicals |
| (C) Atlas White 28190 Oil | Titanium Dioxide (77491) | 22.500 | LCW |
| Dispersible Lot #W05280 | | | |
| (C) Unipure Red LC381 | Iron Oxide (CI77491) | 0.500 | LCW |
| Lot #B7259 | | | |
| (C) Unipure Yellow LC181 | Iron Oxide (CI77492) | 4.400 | LCW |
| Lot #B1946 | | | |
| (C) Unipure Black LC989 | Iron Oxide (CI77499) | 0.165 | LCW |
| Lot #B1273 | | | |

The table below provides an example of a concealer having a “tan” shade.

| Trade Name | INCI Name | % Wt. | Supplier |
|---|---|---|---|
| (A) Jarcol I20 | Octylodecanol | 3.600 | Jarchem |
| (A) Candelilla Wax Regular | Euphorbia Cerifera (Candelilla) | 3.140 | Koster- |
| | Wax | | Keunen |
| (A) Carnauba Wax #1 | Copernica Cerifera (Carnauba) | 3.140 | Koster- |
| | Wax | | Keunen |
| (A) Floraesters 30 | Jojoba Esters | 2.510 | Floratech |
| | | | America |
| (A) DC 200/100 Fluid | Dimethicone | 0.090 | Dow Corning |
| (A) Vitamin E Acetate | Tocopheryl Acetate | 0.090 | BASF |
| (A) Allantoin | Allantoin | 0.050 | ISP |
| | | | Corporation |
| (A) Ascorbyl Palmitate | Ascorbyl Palmitate | 0.050 | DSM |
| NF-FCC | | | Nutritionals |
| (A) Polyethylene 6A | Polyethylene | 1.210 | Honeywell |
| (A) Tixogel CCT6030 | Caprylic/Capric Triglyceride and | 5.140 | Sod-Chemie |
| | Stearalkonium-90 | | Rheologicals |
| | Bentonite and Propylene | | |
| | Carbonate | | |
| (B) Sericite PHN | Mica | 7.300 | Presperse |
| (B) Jarcol 120 | Octyldodecanol | 2.420 | Jarchem |
| (B) Emeressence 1160 | Phenoxyethanol | 1.000 | Cognis |
| (B) SensivaSC50 | Ethylhexylglycerin | 1.000 | Schulke & |
| | | | Mayer |
| (C) Liponate GC | Caprylic/Capric Triglyceride | 43.885 | Lipo |
| | | | Chemicals |
| (C) Atlas White 28190 Oil | Titanium Dioxide (77491) | 18.000 | LCW |
| Dispersible Lot #W05280 | | | |
| (C) Unipure Red LC381 | Iron Oxide (CI77491) | 1.000 | LCW |
| Lot #B7259 | | | |
| (C) Unipure Yellow LC181 | Iron Oxide (CI77492) | 6.000 | LCW |
| Lot #B1946 | | | |
| (C) Unipure Black LC989 | Iron Oxide (CI77499) | 0.375 | LCW |
| Lot #B1273 | | | |

The table below provides an example of a concealer having a “dark” shade.

| Trade Name | INCI Name | % Wt. | Supplier |
|---|---|---|---|
| (A) Jarcol I20 | Octylodecanol | 3.600 | Jarchem |
| (A) Candelilla Wax Regular | Euphorbia Cerifera (Candelilla) | 3.140 | Koster- |
| | Wax | | Keunen |
| (A) Carnauba Wax #1 | Copernica Cerifera (Carnauba) | 3.140 | Koster- |
| | Wax | | Keunen |
| (A) Floraesters 30 | Jojoba Esters | 2.510 | Floratech |
| | | | America |
| (A) DC 200/100 Fluid | Dimethicone | 0.090 | Dow Corning |
| (A) Vitamin E Acetate | Tocopheryl Acetate | 0.090 | BASF |
| (A) Allantoin | Allantoin | 0.050 | ISP |
| | | | Corporation |
| (A) Ascorbyl Palmitate | Ascorbyl Palmitate | 0.050 | DSM |
| NF-FCC | | | Nutritionals |
| (A) Polyethylene 6A | Polyethylene | 1.210 | Honeywell |
| (A) Tixogel CCT6030 | Caprylic/Capric Triglyceride and | 5.140 | Sod-Chemie |
| | Stearalkonium-90 | | Rheologicals |
| | Bentonite and Propylene | | |
| | Carbonate | | |
| (B) Sericite PHN | Mica | 7.300 | Presperse |
| (B) Jarcol 120 | Octyldodecanol | 2.420 | Jarchem |
| (B) Emeressence 1160 | Phenoxyethanol | 1.000 | Cognis |
| (B) SensivaSC50 | Ethylhexylglycerin | 1.000 | Schulke & |
| | | | Mayer |
| (C) Liponate GC | Caprylic/Capric Triglyceride | 44.510 | Lipo |
| | | | Chemicals |
| (C) Atlas White 28190 Oil | Titanium Dioxide (77491) | 12.000 | LCW |
| Dispersible Lot #W05280 | | | |
| (C) Unipure Red LC381 | Iron Oxide (CI77491) | 2.250 | LCW |
| Lot #B7259 | | | |
| (C) Unipure Yellow LC181 | Iron Oxide (CI77492) | 9.600 | LCW |
| Lot #B1946 | | | |
| (C) Unipure Black LC989 | Iron Oxide (CI77499) | 0.900 | LCW |
| Lot #B1273 | | | |

The above formulations are prepared according to the following procedure:

Mix all ingredients in Sequence (A) while heating to 85° C. Bring the temperature of the batch up to 100° C. only long enough to melt the polyethylene. Bring the batch temperature back down to 80° C. and add Sequence (B). Homogenize for five to ten minutes to completely disperse the Tixogel™. Replace the propeller mixer and add Sequence (C). Mix until all color dispersions are completely dispersed and the batch is uniform. If Sequence (C) is added as a complete color grind, premix the pigments into the oil. Roller mill the phase twice and check fineness of the grind on a Hegman Gauge. The reading should be seven or higher on the NS scale (3-5 microns). When free from specks, add the phase to the batch with good continuous mixing. When the batch is on shade, remove the batch and store in airtight containers until ready for filling. The batch should be poured and filled no higher than 68-70° C.

In closing, it is to be understood that the exemplary embodiment described herein are illustrative of the principles of the present invention. Other modifications that may be employed are within the scope of the invention. Thus, by way of example, but not of limitation, alternative configurations may be utilized in accordance with the teachings herein. Accordingly, the description is illustrative and not meant to be a limitation thereof.

What is claimed is:

1. A cosmetic formulation consisting essentially of:
approximately 6% by weight of octylodecanol;
approximately 3.1% by weight of euphorbia cerifera (candelilla) wax;
approximately 3.1% by weight of copernica cerifera (carnauba) wax;
approximately 2.5% by weight of jojoba esters;
approximately 0.09% by weight of dimethicone;
approximately 0.09% by weight of tocopheryl acetate;
approximately 0.05% by weight of allantoin;
approximately 0.05% by weight of ascorbyl palmitate;
approximately 1.2% by weight of polyethylene;
approximately 5.1% by weight of Tixogel®, said Tixogel® comprising caprylic/capric triglyceride and stearalkonium-90 bentonite and propylene carbonate;
approximately 7.3% by weight of mica;
approximately 1% by weight of phenoxyethanol;
approximately 1% by weight of ethylhexylglycerin;
approximately between 40 and 45% by weight of caprylic/capric triglyceride; and
titanium dioxide and iron oxide.

2. The formulation of claim 1, said formulation being a concealer.

3. The formulation of claim 1, said formulation comprising at least 10% by weight of titanium dioxide.

4. The formulation of claim 1, said formulation comprising at least 2% by weight of iron oxide.

5. A cosmetic concealer comprising:
emulsifying agents comprising octylodecanol and caprylic/capric triglyceride, present in an amount of at least 40% by weight;
waxes comprising candelilla and carnauba wax, present in an amount of between approximately 4-8% by weight;
moisturizers and anti-irritants comprising jojoba esters and allantoin, present in an amount of between approximately 1.5-4% by weight;
Skin protectants comprising dimethicone, tocopheryl acetate, ascorbyl palmitate present in an amount of between approximately 0.1-1% by weight; and
Visual effect agents comprising mica and iron oxide.

6. The cosmetic concealer of claim 5, comprising approximately 6% by weight of octylodecanol.

7. The cosmetic concealer of claim 5, comprising approximately 3.1% by weight of euphorbia cerifera (candelilla) wax.

8. The cosmetic concealer of claim 5, comprising approximately 3.1% by weight of copernica cerifera (carnauba) wax.

9. The cosmetic concealer of claim 5, comprising approximately 2.5% by weight of jojoba esters.

10. The cosmetic concealer of claim 5, comprising approximately 0.09% by weight of dimethicone.

11. The cosmetic concealer of claim 5, comprising approximately 0.09% by weight of tocopheryl acetate.

12. The cosmetic concealer of claim 5, comprising approximately 0.05% by weight of allantoin.

13. The cosmetic concealer of claim 5, comprising approximately 0.05% by weight of ascorbyl palmitate.

14. The cosmetic concealer of claim 5, further comprising approximately 1.2% by weight of polyethylene.

15. The cosmetic concealer of claim 5, further comprising approximately 5.1% by weight of Tixogel®, said Tixogel® comprising caprylic/capric triglyceride and stearalkonium-90 bentonite and propylene carbonate.

16. The cosmetic concealer of claim 5, further comprising approximately 7.3% by weight of mica.

17. The cosmetic concealer of claim 5, further comprising approximately 1% by weight of phenoxyethanol.

18. The cosmetic concealer of claim 5, further comprising approximately 1% by weight of ethylhexylglycerin.

19. The formulation of claim 5, said formulation comprising at least 2% by weight of iron oxide.

20. A cosmetic concealer, comprising:
approximately 5-7% by weight of octylodecanol;
approximately 2-4% by weight of euphorbia cerifera (candelilla) wax;
approximately 2-4% by weight of copernica cerifera (carnauba) wax;
approximately 1.5-3.5% by weight of jojoba esters;
approximately 0.05-0.2% by weight of dimethicone;
approximately 0.05-0.2% by weight of tocopheryl acetate;
approximately 0.05-0.2% by weight of allantoin;
approximately 0.05-0.2% by weight of ascorbyl palmitate;
approximately 0.5-2% by weight of polyethylene;
approximately 4-6% by weight of Tixogel®, said Tixogel® comprising caprylic/capric triglyceride and stearalkonium-90 bentonite and propylene carbonate;
approximately 6-8% by weight of mica;
approximately 0.5-1.5% by weight of phenoxyethanol;
approximately 0.5-1.5% by weight of ethylhexylglycerin;
approximately between 40 and 45% by weight of caprylic/capric triglyceride;
at least 10% by weight of titanium dioxide; and
at least 2% by weight of iron oxide.
